(19) United States
(12) Patent Application Publication
(10) Pub. No.: US 2019/0038379 A1
(43) Pub. Date: Feb. 7, 2019

(54) DENTAL RETRACTOR

(71) Applicant: Trustees of Tufts College, Boston, MA (US)

(72) Inventors: Syed ASGHAR, Boston, MA (US); David CHANG, Boston, MA (US)

(21) Appl. No.: 16/053,174

(22) Filed: Aug. 2, 2018

Related U.S. Application Data
(60) Provisional application No. 62/540,609, filed on Aug. 3, 2017.

(51) Int. Cl. A61C 3/00; A61C 5/80; A61B 1/247

(52) U.S. Cl. CPC A61C 3/00; A61B 1/247; A61C 5/80

(57) ABSTRACT

A dental retractor comprises an elongated body having a first axis, a second axis and a third axis, each being orthogonal to each other. The first axis extends longitudinally from a proximal end to a distal end. The first axis and the second axis define a first plane and a width of the elongated body. The first axis and the third axis define a second plane and a thickness of the elongated body. The elongated body includes a blade extending from the distal end and lying substantially in the first plane; and a wing extending from the blade and lying at an angle relative to the first plane.

### CROSS-REFERENCE TO RELATED APPLICATIONS

This application claims the benefit of Provisional Application Ser. No. 62/540,609, filed Aug. 3, 2017, the contents of which are hereby entirely incorporated herein by reference.

### BACKGROUND

This invention relates to covers that are attached to devices, for example, to protect or decorate devices, such as portable electronic devices.

Dentists and oral surgeons use dental retractors to move the cheeks, lips, and tongue out of the way so the mouth and teeth of a patient are frilly exposed. There are a few different types of retractors that dental professionals use to hold specific parts of the mouth aside. For example, a dental retractor can be designed to primarily hold the lips back from the front teeth. A different kind can pull the sides of the mouth, cheeks and flaps aside. When a retractor is used as surgical equipment during oral surgery, it may be used to retract the mucoperiosteal flap. Which is composed of mucous membrane and periosteum.

Dental retractors are generally handheld and some dental retractors are disposable or are made from steel and plastic that can be sterilized and used again.

### SUMMARY

In a general aspect of the invention, a dental retractor comprises an elongated body having a first axis, a second axis and a third axis, each being orthogonal to each other. The first axis extends longitudinally from a proximal end to a distal end. The first axis and the second axis define a first plane and a width of the elongated body. The first axis and the third axis define a second plane and a thickness of the elongated body. The elongated body includes a blade extending from the distal end and lying substantially in the first plane; and a wing extending from the blade and lying at an angle relative to the first plane.

Embodiments of these aspects of the invention may include one or more of the following features.

The blade is configured to retract a buccal or lingual mucoperiosteal flap while the wing is configured to retract the proximal (mesial or distal) mucoperiosteal flap. The blade has a width that tapers to a point at the distal end. The wing is disposed on a first side defined by a first edge of the elongated body. The wing has a width that tapers to a point. The wing includes a serrated edge. The angle of the wing relative to the blade is in a range between 90 and 130 degrees, preferably 110 degrees. The width is in a range between 10 and 20 mm, preferably 15 mm. The length of the blade is in a range between 5 and 15 mm, preferably 10 mm. The length of the wing is in a range between 5 and 15 mm, preferably 10 mm. The proximal end of the elongated body further includes a second blade and a second wing (for use on the opposite side of the maxillary or mandibular dental arch).

The wing may include an edge having serrations configured, for example, to engage the surface of the bone and serve as a stop which will prevent apical slippage of the blade and undue pivoting rotation of the dental retractor.

The dental retractor further includes a handle between the proximal end and distal end. The handle is in a third plane offset from the first plane to improve field of view of the distal end of the retractor during use. The elongated body is formed of stainless steel.

In another general aspect of the invention, a method comprises exposing underlying bone or a portion of a tooth including making a first incision to form a first mucoperiosteal flap; making a second incision to form a second mucoperiosteal flap; and retracting the first mucoperiosteal flap and the second mucoperiosteal flap simultaneously using a dental retractor.

Embodiments of this aspect of the invention may include the first mucoperiosteal flap being a buccal or lingual mucoperiosteal flap and the second mucoperiosteal flap being a proximal (mesial or distal) mucoperiosteal flap.

A dental retractor and its method of use provide numerous advantages. In particular, the dental retractor is configured and structured to retract more than one mucoperiosteal flap at the same time. Being able to retract more than one flap with a single instrument, advantageously permits better visibility of the interface between the tooth and the underlying bone without crowding the surgical site with more instruments. Retraction of the mucoperiosteal flap may be desirable for surgical procedures involving the jaw bones including extraction of a tooth (e.g., wisdom tooth), accessing a tumor or cyst on the underlying bone, biopsy at the bone, or replacing a tooth with a dental implant.

Where multiple instruments are used during the procedure (e,g., drill, retractor, suction, etc.), the dental practitioner or person assisting the dental practitioner has greater flexibility to assist since at least one less instrument is not needed.

Moreover, by retracting multiple flaps simultaneously in the mandibular arch, the procedure can be performed more quickly and, risk of lingual nerve damage is minimized as fewer instruments in the surgical area will prevent iatrogenic injury to the nerve, which lies in anatomical proximity to the mandibular bone.

Other features and advantages of the invention are apparent from the following description, and from the claims.

### DESCRIPTION OF DRAWINGS

FIG. 1 is top view of the mandibular teeth of a patient.

FIG. 2 is a schematic, partially cross-sectional, perspective view of a representative teeth and surrounding tissue

FIG. 3 is a perspective view of a dental retractor.

FIG. 4 is a top view of the dental retractor of FIG. 3.

FIG. 5 is a side view of the dental retractor of FIG. 3,

FIG. 6 is a perspective view of one working end of the dental retractor of FIG. 3.

FIG. 7 is an end view of one working end of the dental retractor of FIG. 3.

FIG. 8 shows the dental retractor being used to retract a buccal and distal periodontal flap.

### DESCRIPTION

As will be described in greater detail below, a dental retractor for retracting soft tissue of the jaw improves visibility and access to the underlying bone. More particularly, the soil tissue to be retracted by the dental retractor is the periodontal full-thickness flap (i.e., mucoperiosteal flap) that is surgically separated from the underlying tissue to provide access to the bone and root surface. There are a variety of reasons for requiring visibility of that part of the tooth anatomy such that retraction of the mucoperiosteal flap is desirable. For example, retraction of the mucoperiosteal flap may be desirable for procedures including extraction of a tooth (e.g., wisdom tooth), accessing a tumor or cyst on the underlying bone, biopsy at the bone, or replacing a tooth with a dental implant.

Before delving into the details of the structure for achieving improved visibility, a brief description of the anatomy of a human's teeth and surrounding tissue is appropriate.

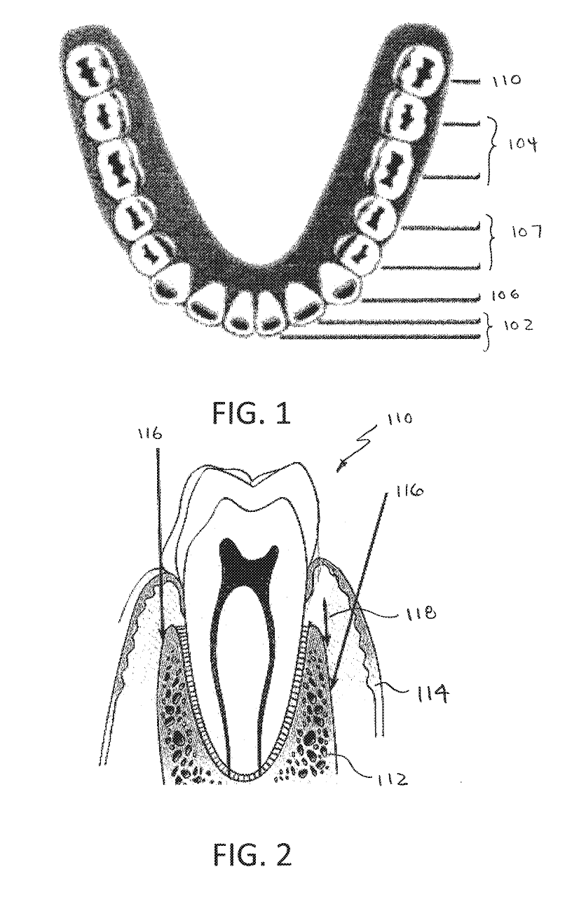

Referring to FIG. 1, mature humans have teeth 100 that are arranged in the mouth such that incisors 102 are in the anterior of the mouth, molars 104 are in the posterior of the mouth. Canines 106 and premolars 107 are positioned between incisors 102 and molars 104. Wisdom teeth 110 are positioned adjacent the rearward-most molar.

Referring to FIG. 2, a representative tooth 110 is disposed within depressions formed within underlying bone 112. The lower part of the tooth including its root as well as the underlying bone is covered by gum 114. The interface between the gum 114 and hone 112 is lined with the periosteum 116. To provide for the visibility and access to the hone 112 and root surface of tooth 110, incisions into the soft tissue of gum 114 are made by the dental practitioner. The incisions form periodontal flaps (see FIG. 8), which are soft tissue raised off the bone. For example, an incision can be made along arrow 118. These periodontal flaps can be identified by the side of the tooth that is being accessed. Specifically, from a dentist's point of view looking at a patient's lower teeth, the periodontal flaps include:
a. Mesial flap: the flap toward the dentist;
b. Distal flap: the flap toward the throat
c. Buccal flap: the flap toward the cheek
d. Lingual flap: the flap toward the tongue.

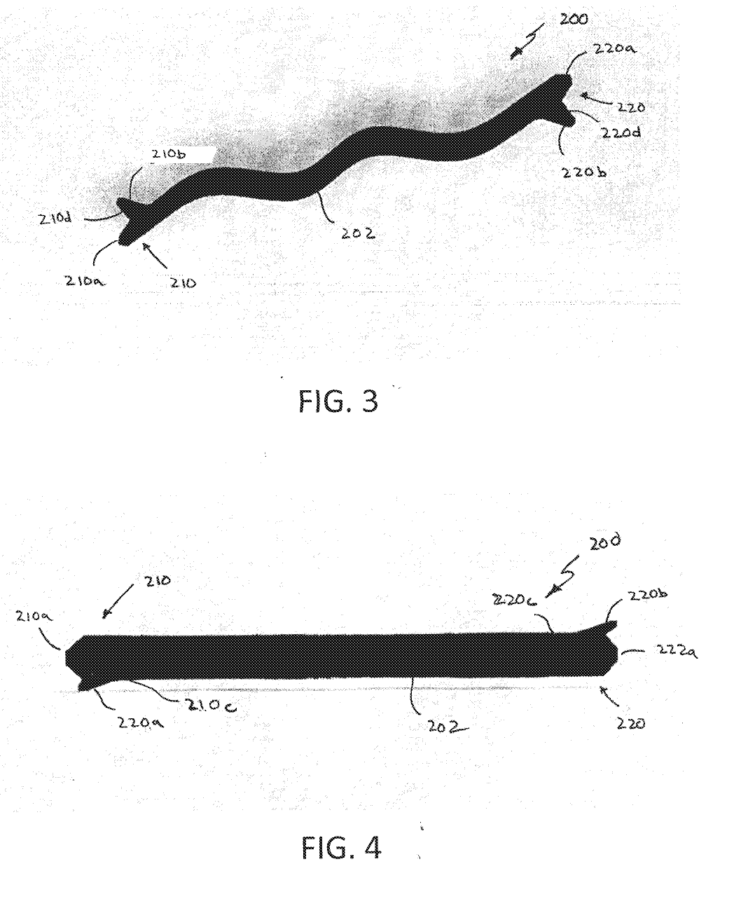

Referring to FIGS. 3, 4, and 5, a dental retractor 200 for retracting flaps to allow better visualization of the surgical site is shown. Dental retractor is in the form of an elongated body 202 formed of a rigid material (e.g., stainless steel) having the following general dimensions:
a. Length: 150 to 200 mm (e.g., 180 mm)
b. Width: 10 to 25 mm (e.g., 15 mm)
c. Thickness: 1.0 to 3 mm (e,g., 2 mm).

For reasons discussed in greater detail below, elongated body 202 is wavy in shape. Elongated body 202 has working ends 210 and 220 at either end with the intermediate portion of the body serving as a handle for the dentist.

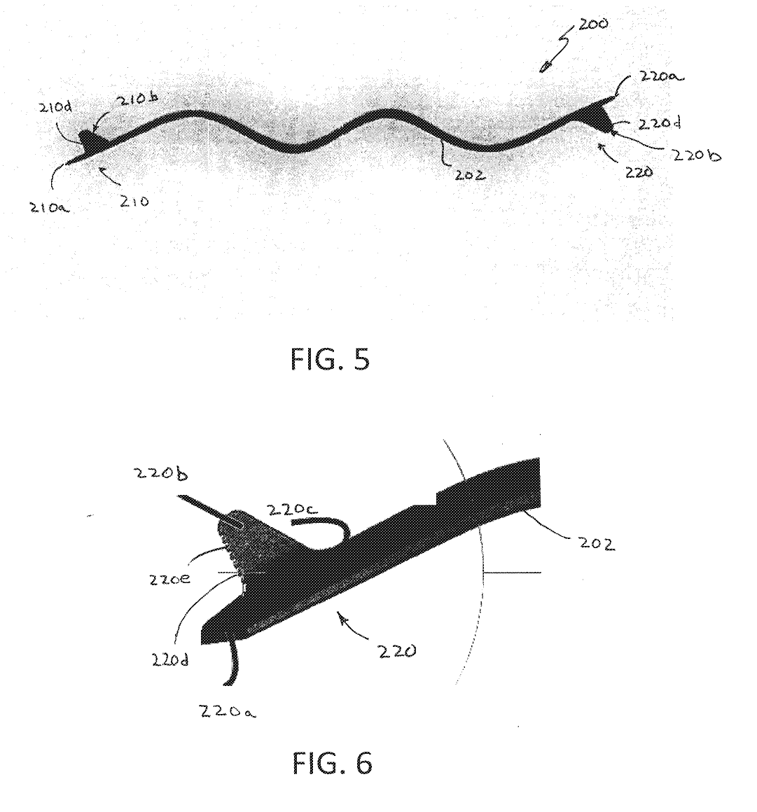

Referring to FIGS. 5 and 6, working ends 210, 220 both include a blade 210a, 220a at respective ends of elongated body 202 to retract a mucoperiosteal flap after incision. Blades 210a, 220a taper in two directions. For example, in an embodiment in which the thickness of elongated body 202 is 2 mm thick, blades 210a, 220a taper to a thickness of about 1 mm or less. And, in the embodiment in Which the width of elongated body 202 is 15 mm, blades 210a, 220a taper for a length in a range of 5 mm to 10 mm (e.g., 7 mm) to an end width of about 5 mm or less.

Working ends 210, 220 also include a wing 210b, 220b at respective ends of elongated body 202 to retract the proximal (mesial or distal) flap after incision. Wings 210b, 220b have a thickness of about 1 to 2 mm and lengths of about 5 mm to 15 mm (e.g., 10 mm) when measured from the surface of elongated body 202. Wings 210b, 220b are also tapered as they extend away from the surface of elongated body and have a width of about 10 mm to a rounded end having a width of about 3 mm. Distal surfaces 210d, 220d of wings 210b, 220b may include serrations 220e to provide better traction with the surface of the bone adjacent to where the flap attaches to bone (see FIG. 8). As shown in FIG. 6, the junction between the blade 210a and the wing 210b permits for the serrations 220e of the wing to engage the surface of the bone and serve as a stop which will prevent apical (towards the roots of the teeth) slippage of the blade and undue pivoting rotation of the instrument, thus enhancing stability and preventing iatrogenic injury.

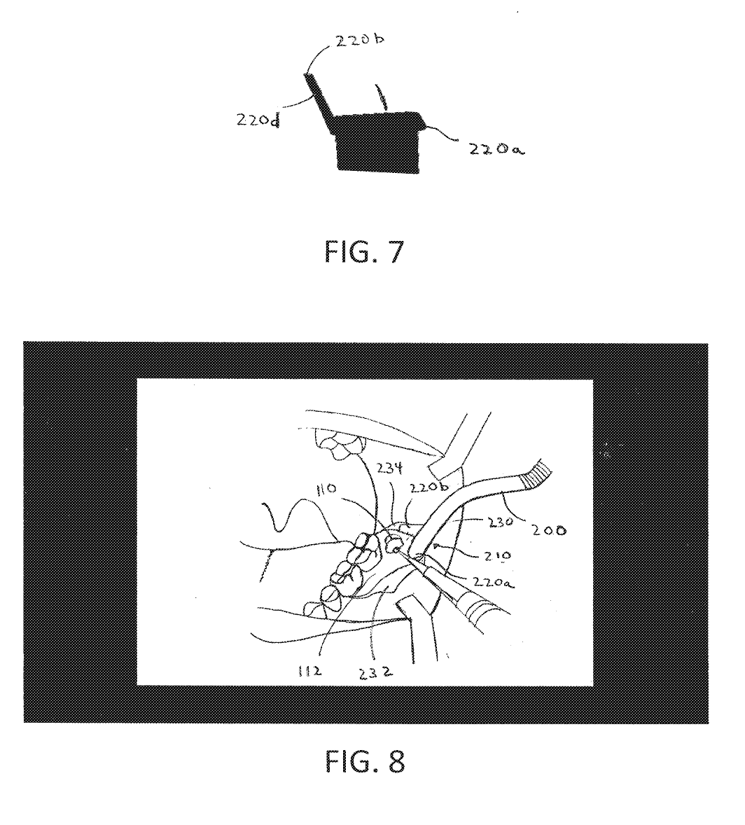

As shown best in FIG. 7, wings 210b, 220b have a generally right-angle shape and extend at an angle from a side surface 210c, 220c of elongated body 202 in a range from 90-120 degrees (e.g., 110 degrees).

With this arrangement, dental retractor 200 serves as a single instrument that is capable of retracting two mucoperiosteal flaps at the same time. For example, dental retractor can retract or draw both of the buccal and distal flaps away from a tooth. In this way, the dentist is much improved visibility of the portion of the tooth where he is likely to use other instruments for the surgical procedure.

As can be seen from the embodiment shown in FIGS. 3-7., the two working ends 210, 220 of dental retractor 200 are used for retracting mucoperiosteal flaps.

For example, referring to FIG. 8, if the dental practitioner needs to access wisdom tooth 110 impacted in bone 112 located in the lower left-side of a patient's mouth, working end 220 (comprised of blade 220a and wing 220b) would be used to retract buccal flap 232 and distal flap 234 of that tooth. In particular, blade 220a would retract buccal flap 232 while wing 220b would retract distal flap 234.

Note that if the dental practitioner now needs to extract the wisdom tooth located in the lower right-side of the patient's mouth, she would invert dental retractor 200 so that working end 210 can be used to retract the buccal and distal flaps of the wisdom tooth at the other side of the mouth.

Although dental retractor 200 is particularly advantageous in retracting mucoperiosteal flaps associated with the mandibular teeth, the dental retractor can also be used to retract the flaps associated with the maxillary teeth. It is also important to appreciate that dental retractor 200 can also be used for any segment of the jaws where there is sufficient space for the dental retractor to access the distal or mesial flap.

Other embodiments are within the scope of the following claims. For example, dimensions relating to the size, shape, and relative dimensions (length, angle) of the dental retractor as well as its blades and wings can be varied in accordance with the particular area of underlying bone being accessed. The size, location and type of teeth are factors in determining which type of retractor is to be used.

What is claimed is:

1. A dental retractor comprising:
an elongated body having a first axis, a second axis and a third axis, each being orthogonal to each other, the first axis extending longitudinally from a proximal end to a distal end, the first axis and the second axis defining a first plane and a width of the elongated body, the first axis and the third axis defining a second plane and a thickness of the elongated body, the elongated body including:
a blade extending from the distal end and lying substantially in the first plane; and
a wing extending from the blade and lying at an angle relative to the first plane.

2. The dental retractor of claim 1 wherein the blade is configured to retract a buccal mucoperiosteal flap.

3. The dental retractor of claim 1 wherein the blade is configured to retract a lingual mucoperiosteal flap.

4. The dental retractor of claim 1 wherein the wing is configured to retract a mesial mucoperiosteal flap.

5. The dental retractor of claim 1 wherein the wing is configured to retract a distal mucoperiosteal flap.

6. The dental retractor of claim 1 further comprising a handle between the proximal end and distal end.

7. The dental retractor of claim 6 wherein the handle is in a third plane offset from the first plane to improve field of view of the distal end of the retractor during use.

8. The dental retractor of claim 1 wherein the wing is disposed on a first side defined by a first edge of the elongated body.

9. The dental retractor of claim 1 the blade has a width that tapers at the distal end.

10. The dental retractor of claim 1 wherein the wing has a width that tapers at the distal end.

11. The denial retractor of claim 1 wherein the wing includes a serrated edge.

12. The dental retractor of claim 1 wherein the elongated body is formed of stainless steel.

13. The dental retractor of claim 1 wherein the angle of the wing relative to the blade is in a range between 90 and 130 degrees, preferably 110 degrees.

14. The dental retractor of claim 1 wherein the width is in a range between 10 and 20 mm, preferably 15 mm.

15. The dental retractor of claim 1 wherein the length of the blade is in a range between 5 and 15 mm, preferably 10 mm.

16. The dental retractor of claim 1 wherein the length of the wing is in a range between 5 and 15 mm, preferably 10 mm.

17. The dental retractor of claim 1 wherein the wing includes an edge having serrations configured, for example, to engage the surface of the bone and serve as a stop which will prevent apical slippage of the blade and undue pivoting rotation of the dental retractor.

18. A method comprising:
exposing a portion of a bone or tooth including:
making a first incision to firm a first mucoperiosteal flap;
making a second incision to form a second mucoperiosteal flap; and
retracting the first mucoperiosteal flap and the second mucoperiosteal flap simultaneously using a dental retractor.

19. The method of claim 18 wherein the first mucoperiosteal flap is a buccal or lingual flap and the second mucoperiosteal flap is a mesial or distal mucosa flap.

20. The method of claim 18 wherein the dental retractor comprises an elongated body having a first axis, a second axis and a third axis, each being orthogonal to each other, the first axis extending longitudinally from a proximal end to a distal end, the first axis and the second axis defining a first plane and a width of the elongated body, the first axis and the third axis defining a second plane and a thickness of the elongated body, the elongated body including a blade extending from the distal end and lying substantially in the first plane; and a wing extending from the blade and lying at an angle relative to the first plane.
